(19) United States
(12) Patent Application Publication
(10) Pub. No.: US 2020/0122134 A1
(43) Pub. Date: Apr. 23, 2020

(54) EFFICIENT CAPTURE OF MAGNETIC AND PARAMAGNETIC CARRIERS OF BIOMARKERS FROM A FLOWING SYSTEM

(71) Applicant: Vanderbilt University, Nashville, TN (US)

(72) Inventors: Frederick R. HASELTON, Nashville, TN (US); Stephanie I. PEARLMAN, Nashville, TN (US)

(73) Assignee: Vanderbilt University, Nashville, TN (US)

(21) Appl. No.: 16/656,658

(22) Filed: Oct. 18, 2019

Related U.S. Application Data
(60) Provisional application No. 62/748,108, filed on Oct. 19, 2018.

(51) Int. Cl. B01L 3/02; G01N 33/543; C12Q 1/6806; C12N 15/10; C07K 1/14

(52) U.S. Cl. CPC B01L 3/021; G01N 33/54326; C12Q 1/6806; C12N 15/1013; B01L 2200/16; B01L 2300/047; B01L 2300/12; B01L 2300/06; B01L 2300/0832; C07K 1/145

(57) ABSTRACT

A device and method for performing low resource processing of biological or environmental samples is disclosed. The device uses high gradient magnetic separation to manipulate magnetic or paramagnetic beads for capture and isolation of analytes (proteins or nucleic acids) from solution. A disposable transfer pipette with tip containing a copper, aluminum or steel matrix is used to i) remove capture agent-coated magnetic beads bound to sample nucleic acids or proteins from the initial sample, ii) expose the analyte-bead complex to successive processing solutions and iii) separate the concentrated analytes from the beads in the final elution step.

### PRIORITY CLAIM

This application claims benefit of priority to U.S. Provisional Application Ser. No. 62/748,108, filed Oct. 19, 2018, the entire contents of which are hereby incorporated by reference.

The invention was made with government support under Grant No. D43TW009348 awarded by the National Institutes of Health. The government has certain rights in the invention.

### BACKGROUND

Paramagnetic micron size beads are often used to separate materials from a suspension. In biotechnology applications they have been increasingly used to capture a specific biomarker or cell. In most applications this is accomplished by holding an external magnet next to a tube and then pouring out or removing the liquid. Flow though recovery of these paramagnetic beads is not used, primarily because holding a magnet next to a suspension of beads does not efficiently capture the beads as they flow by the magnet.

### SUMMARY

Thus, in accordance with the present disclosure, there is provided a kit comprising (a) a transfer pipette comprising a reservoir end and an open tip end, wherein the reservoir end of said pipette contains magnetic or paramagnetic beads coated with an analyte binding reagent, and wherein the open end of said pipette comprises steel, copper or aluminum matrix in its lumen; and (b) a magnet. The kit may further comprise one or more of:

(c) a syringe comprising a glass wool-filled tip;

(d) one or more sample collection tubes;

(e) one or more containers comprising processing reagents; and/or

(f) a tuberculin syringe comprising a glass wool filter.

The one or more containers may contain a dilution buffer, a binding buffer, one or more wash solutions, a rinse solution and/or an elution solution. The kit may further comprise a reagent for performing positive and/or negative control reactions. The kit may further comprise a device for performing sample collection, such as a a syringe, a cup, a tube, a spatula or needle. The steel, copper or aluminum matrix may be transiently magnetized. The kit may further comprise printed instructions for performing sample processing using said kit. The magnetic beads may be magnetic or paramagnetic silica beads. The analyte binding reagent may be a nucleic acid, antibody, lipid, carbohydrate, or non-antibody protein that binds a target analyte.

In another embodiment, there is provided a transfer pipette comprising a reservoir end and an open tip end, wherein the reservoir end of said pipette contains magnetic or paramagnetic beads coated with an analyte binding reagent, and wherein the open end of said pipette comprises steel, copper or aluminum matrix in its lumen. The matrix may be magnetized. The magnetic or paramagnetic beads may be magnetic or paramagnetic silica beads. The analyte binding reagent may be a nucleic acid, antibody, lipid, carbohydrate, or non-antibody protein that binds a target analyte. The analyte may be a viral, fungal, bacterial or parasitic antigen or an environmental toxin. The bacterial antigen/nucleic acid may be derived from Mycobacterium tuberculosis. The viral antigen/nucleic acid may be derived from Ebola virus, dengue virus, Rift River Valley virus, West Nile virus, Marburg virus, rabies virus, HIV-1, HIV-2, smallpox, hantavirus, influenza virus, Zika virus or rotavirus. The fungal antigen/nucleic acid may be derived from Candida, Histoplasma, Blastomyces, Cryptococcus, Coccidioides, or Paracoccidioides. The parasitic antigen/nucleic acid may be derived from Plasmodium spp., tapeworm, ringworm, heartworm, roundworm, ascaris, whipworm, toxocara, and Acanthamoeba. The transfer pipette may further comprise lyophilized reagents for performing a binding or detection assay.

In yet another embodiment, there is provided a method of purifying an analyte from a solution comprising (a) providing a biological or environmental sample; (b) introducing said sample into a transfer pipette, wherein said transfer pipette comprises a reservoir end and an open tip end, wherein the reservoir end of said pipette contains magnetic or paramagnetic beads coated with an analyte binding reagent, and wherein the open end of said pipette comprises steel, copper or aluminum matrix in its lumen; (c) expelling the sample from said pipette into a first receptable while placing a magnetic adjacent to said open tip of said pipette; (d) introducing a first wash solution into said transfer pipette after removing the magnet; (e) expelling the first wash solution into a second receptacle while:

the tip of said pipette is inserted into said second receptacle; and

(ii) the magnet is placed adjacent to said second receptable,

(f) introducing elution solution into said transfer pipette after removing the magnet; and (g) expelling the elution solution into a third receptacle.

The analyte may be a protein, a nucleic acid, a carbohydrate, a chemical toxin, lipid, virus, parasite, bacteria. The protein or nucleic acid may be a viral, bacterial, fungal or parasite protein or nucleic acid. The analyte binding reagent may be a nucleic acid, antibody, lipid, carbohydrate, or non-antibody protein that binds a target analyte. The sample may be a biological sample such as sputum, saliva, urine, blood or blood product, stool, tears, or spinal fluid. The magnetic or paramagnetic beads may be magnetic or paramagnetic silica beads. The method may further comprise obtaining said sample from a subject or an environment. The sample may be pre-processed prior to step (b) to dilute, purify, filter and/or decontaminate said sample.

The method may further comprise performing a second wash following step (e) and comprising (e′) introducing a second wash solution into said transfer pipette after removing the magnet; (e″) expelling the second wash solution into a fourth receptacle while:

(i) the tip of said pipette is inserted into said fourth receptacle; and

(ii) the magnet is placed adjacent to said fourth receptable.

The first wash solution may be a chaotropic wash solution and the second wash solution is an alcohol wash solution. The method may further comprise performing a rinse step following step (e) and comprising (e′″) introducing a rinse solution into said transfer pipette after removing the magnet; (e″″) expelling the rinse solution into a fifth receptacle while:

(i) the tip of said pipette is inserted into said fifth receptacle; and

(ii) the magnet is placed adjacent to said fifth receptable.

Steps (b), (d), (e′), (e′″) and/or (f) may further comprise ejecting and re-introducing the relevant solution into the pipette multiple times. The method may further comprise analyzing the magnetic or paramagnetic beads in the elution solution produced by step (g) for the presence of said analyte. The method may be performed in less than 5 minutes and/or achieve 100% recovery of said magnetic or paramagnetic beads. The elution solution may be composed of two immiscible liquids, and in only one of said immiscible liquids the analyte is miscible, thereby concentrating the eluted analyte into a single phase.

The use of the word “a” or “an” when used in conjunction with the term “comprising” in the claims and/or the specification may mean “one,” but it is also consistent with the meaning of “one or more,” “at least one,” and “one or more than one.” The word “about” means plus or minus 5% of the stated number.

It is contemplated that any method or composition described herein can be implemented with respect to any other method or composition described herein. Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following drawings form part of the present specification and are included to further demonstrate certain aspects of the present disclosure. The disclosure may be better understood by reference to one or more of these drawings in combination with the detailed description of specific embodiments presented herein.

FIG. 1. Kit components.

FIG. 2. Example of high gradient magnetic separation of 1-micron magnetic beads from a flow using a small steel wool plug (arrow) and an external magnet (left panel); change in the appearance of a magnetic bead suspension after separation (right panel).

FIG. 3. Comparison of sample processing times for stool, sputum and whole blood with critical timing steps (in red).

FIG. 4. Effect of external magnet on the capture of 1 micron paramagnetic beads showing that an external magnetic is required for capture. Flow rate=18.45+/−0.28 mL/min.

FIG. 5. Steel wool+magnetic field captures nearly 100% over a wide range of flows.

FIG. 6. Viscosities up to 55 cP do not have a significant effect on capture. Effect of viscosity on 1 μm bead capture in a high-gradient magnetic separator. Viscosity measurements were taken at a strain rate of 37.5 s⁻¹. One-way ANOVA or unpaired t-test performance for statistical analysis. Blue: glycerol solutions of increasing viscosity (p>0.05). Black: synthetic sputum before and after liquefaction with guanidine thiocyanate buffer (p>0.05).

FIG. 7. Surface fields less than 1708 Gauss results in significantly reduced capture. Flow rate=19.86+/−1.42 mL/min.

FIG. 8. Capture increases with mass of steel wool to a maximum.

FIG. 9. Steel wool diameter has little effect on capture, but release depends on diameter.

### DETAILED DESCRIPTION

This technology describes a design to concentrate nucleic acid biomarkers from much larger volumes of urine by capturing magnetic beads from a flow using a “magnetic frit.” In order to re-collect the magnetic beads from a flow, increased magnetic capture from the flow is achieved by inserting a small piece of non-magnetic steel wool or other non-magnetic matrix material that is transiently magnetized by an external magnet. A second characteristic of this magnetic frit design is that the steel wool or other transiently magnetizable matrix material remains magnetized only when the external magnet is present. When the external magnet is removed, 100% of the paramagnetic beads are readily flushed out of the steel wool matrix. One important feature is that steel wool matrix captures biomarkers bound to the surface of paramagnetic beads from a flowing system.

### I. THE SYSTEM

The inventor has developed a simple design to concentrate nucleic acid biomarkers from, e.g., much larger volumes of urine by capturing magnetic beads from a flow using a “magnetic frit.” Much of the inventors' previous work using magnetic beads and magnetics was focused on enabling the processing of magnetic beads through stationary fluids. In examining the inverse problem, that is how to re-collect the magnetic beads from a flow, the inventor considered increasing the magnetic capture from the flow by inserting a small piece of non-magnetic steel, aluminum or copper wool matrix that is transiently magnetized by an external magnet (see Ge et al., Results Physics 7:4278-4286, 2017 for a discussion of magnetic matrices). This completely solved the re-collection problem and the inventors capture 100% of the paramagnetic magnetic beads. Importantly, the phenomenon is so robust that for any user produced flows using a simple transfer pipette, re-collection is always 100%. In other words, this design reduces the potential error due to a variation in flow. Interestingly, further searching showed this phenomenon has been described in the 1970s for mining and sewage applications as a means to capture weakly paramagnetic materials such as CuO from a flowing mine slurry or to remove paramagnetic contaminants from flowing sewage or water supplies.

Moreover, the system described herein employs magnetized steel wool matrix as a capture material and is very efficient at bead capture and appears to be near 100%. Furthermore, when the external magnet is removed the steel wool matrix is no longer magnetic and the paramagnetic beads can be completely recovered. If the external magnetic field is retained, additional processing steps are enabled with high efficiency since the beads are well-dispersed and not compacted against the side of the suspension container.

### II. DEVICE FEATURES

The device uses a single inlet and outlet. Because the device is to be implemented in a low-resource setting, the use of a single inlet and outlet eliminates the need for external pumps and fluidics that some systems use as the sample can be contained within the device. This also helps eliminate cross-contamination from infectious materials. The outflow through the ferromagnetic matrix is controlled entirely by the user. Other dual inlet/outlet HGMS systems make use of a gravity feed system, which does not allow the user to control the rate of flow through the system or requires the use of extra components to control the flow rate. For example, some systems use gravity feed, and requires the user to control flow rate by inserting a needle at the bottom of the column, with smaller gauges resulting in a lower flow rate. While this is generally effective, it does not allow for total control of the outflow as our device would. In addition, parts such as these present a risk to the user, as accidental punctures and stick to the user are possible; with users already working without proper PPE or containment while handling potentially infectious materials, the addition of another possible exposure source is not acceptable.

This system is meant to be single use to maintain sterility and prevent contamination between samples, and prior art discusses the use of cleaning and reuse of 1) the column, 2) the magnetic seeds/particles, or 3) reuse of both. It is also common to have personnel working with significantly less training than is required in developed countries, meaning that the system needs to be as simple to use and be essentially fail-proof, making the single inlet-outlet favorable. This device will not need to be cleaned or prepped before use, decreasing the demand requirements of use for the user.

One modification to the device allows for use of a paramagnetic matrix in place of a ferromagnetic matrix used in the prior art. Ferromagnetic materials experience a hysteresis effect, and therefore have a residual magnetism that may influence the release of the paramagnetic particles in the system. Paramagnetic materials do not experience this, eliminating the concern of residual magnetism negatively affecting bead recovery from the device. In addition, paramagnetic materials do not saturate in a magnetic field in the way that ferromagnetic materials do, so there is no limit on the strength of the induced gradients within the matrix.

Previous approaches showed that to achieve >90% recovery of beads from a matrix, it must be flushed with multiple column after the fact. However, lab equipment such as centrifuges, and dehydrators, which would commonly be used for sample concentration, are not available to personnel in low-resource settings due to a variety of factors, including but not limited to electricity access, personnel training, and economic factors. Therefore, it is imperative that a device has the ability to concentrate particles in a volume significantly smaller than the original sample. To perform this, a release method that makes use of the hydrophobicity of oil and water to encourage release is being employed. Using a large volume of oil will provide the volume needed to remove beads from the matrix, while the aqueous phase captures the beads due to hydrophilic interactions between the beads and water. These proportions could also be reversed if the particles or released materials are hydrophobic, instead capturing in the oil while flushing the matrix with water. Also, the single inlet-outlet mentioned previously allows the sample volume of eluate to be passed through the matrix multiple times, something that is difficult to achieve with pump or gravity driven systems containing a separate inlet and outlet. This also allows for a much smaller release volume to be used, unlike the large volumes commonly used in prior art.

While prior efforts have used coatings to prevent damage to the sample (e.g., cells), or prevent corrosion of the wire, the coating is not used for this purpose, but for the purposes of optimizing the release of particles from the device. With a coating that yields chemically unfavorable interactions (e.g., ionic, hydrophobic interactions) between the particles and matrix, one can further improve release characteristics on the molecular scale in addition to the bulk device effects.

### III. SAMPLES AND ANALYTES

Samples for use in the present disclosure include any biological sample, include blood, blood products (serum, plasma), sputum, saliva, mucous, stool, urine, ascites, tears, or pus. Samples may also be environmental, such as water, soil, plant extracts, industrial waste or food stuffs.

Samples may be obtained from an environment or a subject as part of the disclosed methods. Methodologies for sample collection are well known in the art. The method may also start with previously obtained samples, optionally such as those already pre-processed.

The samples will normally be those suspected of containing analytes of interest. These include whole or part of bacteria, viruses, fungi or parasites, including proteins and nucleic acids. They may also contain chemical species, such as drugs, toxins, industrial by products, herbicides, insecticides, etc.

### IV. EXAMPLES

The following examples are included to demonstrate preferred embodiments. It should be appreciated by those of skill in the art that the techniques disclosed in the examples that follow represent techniques discovered by the inventor to function well in the practice of embodiments, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1

In the current format, the protocol is not automated. However, the long-term goal of this project is to create a new kit for nucleic acid extraction that does not require micropipettes for liquid handling or extensive skills to operate. This kit would include all solutions premade in aliquots, eliminating the need for micropipettes.

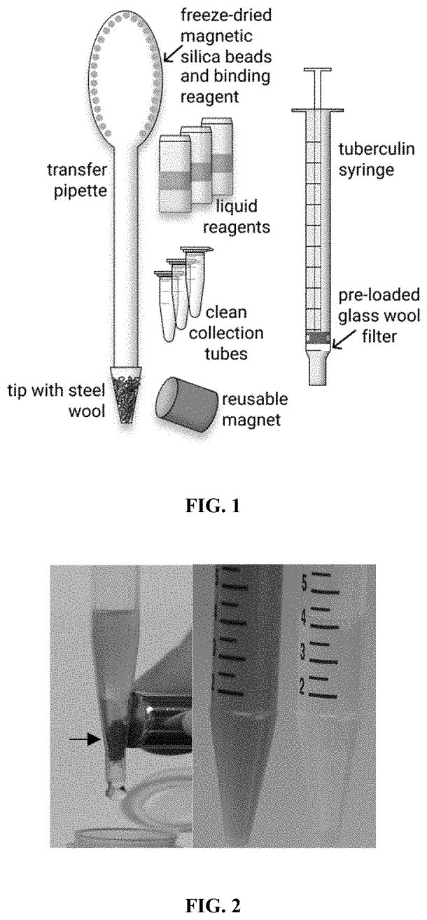

The main features include:
all steps are performed with a transfer/bulb pipet
inexpensive components
steel, copper, aluminum wool or other transiently magnetizable material is incorporated to maximize bead capture and recovery
incorporates enhanced sample mixing
potential for assembled and pre-packaged reagents
   The device uses high gradient magnetic separation to manipulate silica-coated magnetic beads for capture and isolation of nucleic acids from solution. A disposable transfer pipette with tip containing stainless steel wool (FIG. 1) is used to i) remove silica-coated magnetic beads bound to sample nucleic acids from the initial sample matrix, ii) expose the nucleic acid-bead complex to successive processing solutions and iii) separate the concentrated nucleic acids from the magnetic beads in the final elution step. This format also facilitates simple mixing and enhances the interaction between the surface-bound nucleic acids and the processing solutions. The basic principle of operation is based on a high gradient magnetic separation phenomenon which creates a high gradient magnetic field when an external magnetic field is applied, resulting in ˜100% capture and concentration of magnetic beads (FIG. 2). Conveniently, the entire nucleic acid-bead complex can also be removed transiently by removing the external magnet to release the complex from the steel wool. The end result is a flexible and highly efficient platform for purifying nucleic acids from complex sample matrices.

Nucleic acids are captured with silica-coated magnetic beads using traditional silica-capture nucleic acid chemistries and a high gradient magnetic separator in a transfer pipette as a processor.

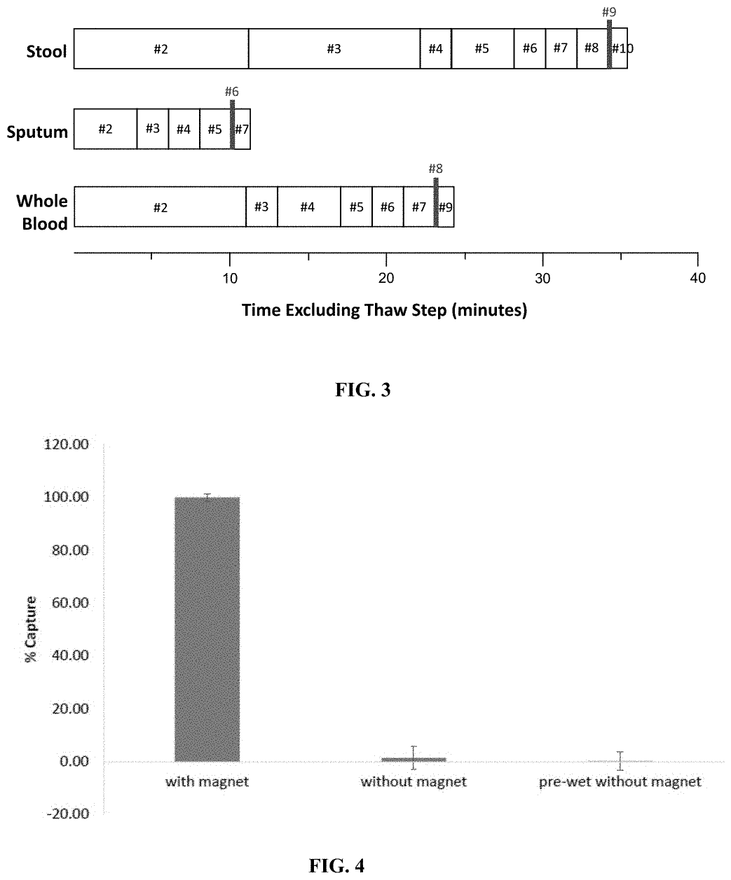

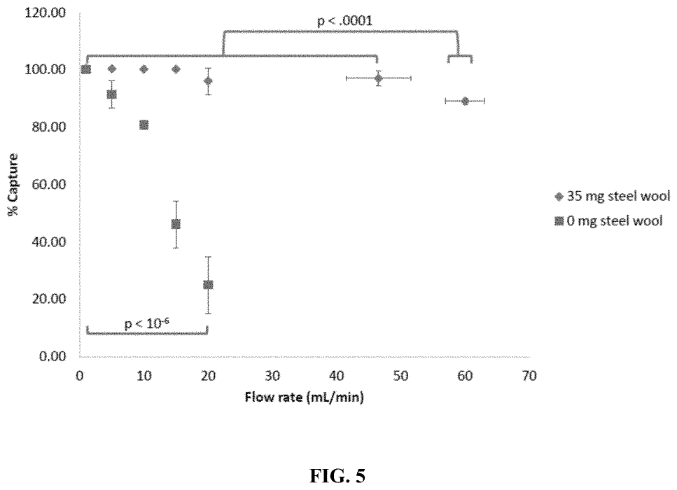

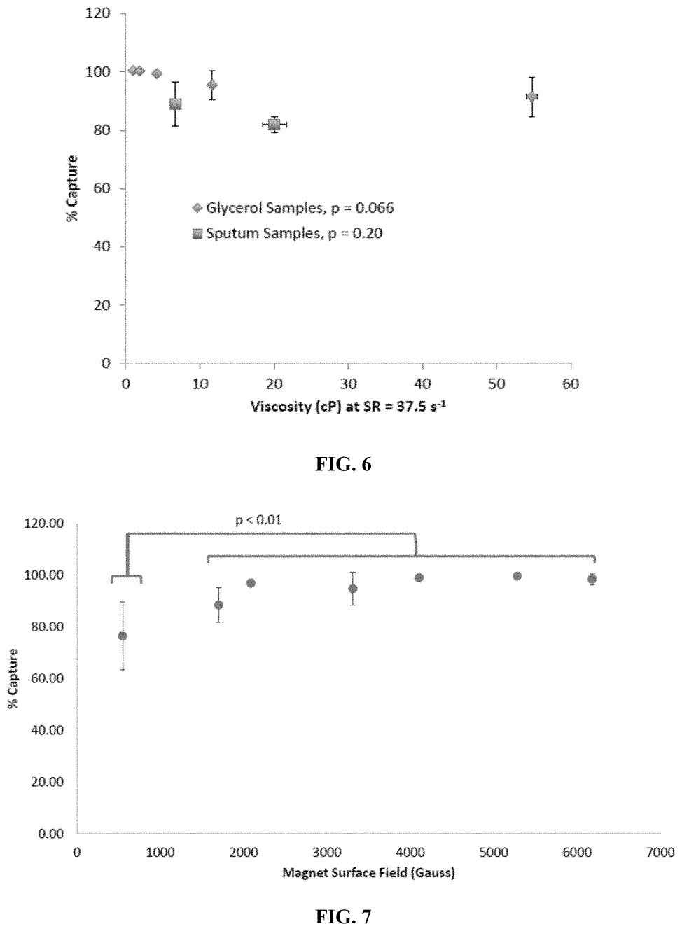

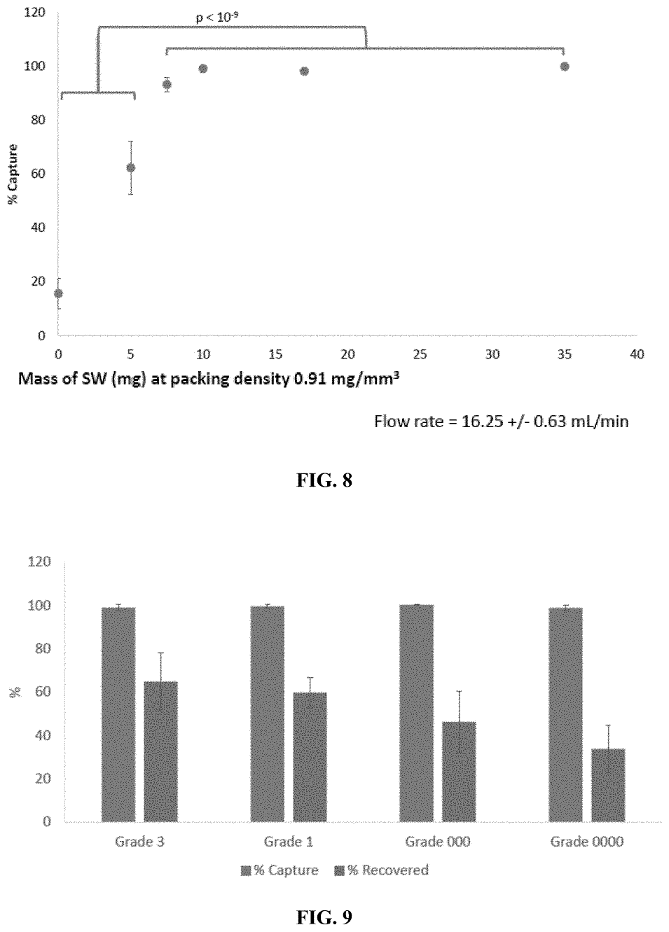

The design goal was not to use any electricity, but in this evaluation the inventor used a centrifuge to remove particulate matter from the frozen stool samples and a heat block to increase RNA recovery from the frozen blood samples. It is anticipated that with more development to be able to eliminate these electricity-requirements, particularly for non-frozen samples. The following materials and workflow were employed and FIG. 3 indicates critical timing steps:

Kit Components and Processing Solutions:
Transfer pipette: 3.2 mL (Fisher 13-711-7)
Pipette tip: 1-200 μL (Fisher 02707505)
Steel wool: 18 mg Type 434 stainless steel wool grade (˜50-micron diameter)
Processing Magnet: K&J Magnetics, # B666-N52; ⅜ in cube
Tuberculin syringe pre-loaded with small plug of glass wool
Beads: Dynabeads MyOne Silane (Thermo Fisher Cat #37002D)

Protocols were Developed Using the Following Solutions:
Binding Buffer: 4M guanidine thiocyanate, 10 mM Tris HCl (pH 8), 1 mM EDTA (pH 8), 0.5% Triton X-100
Chaotropic Wash Buffer: 84% ethanol, 640 mM guanidine thiocyanate, 1.6 mM Tris HCl (pH 8), 160 mM EDTA (pH 8)
Ethanol Wash: 70% Ethanol
Rinse Solution: TE buffer, pH 8
Elution Solution: TE buffer, pH 8
Protocol-specific additives: ß-mercaptoethanol, isopropanol, proteinase K (>600 mAU/mL), phosphate buffered saline

Frozen Stool—Process & Turnaround Time. Process Time for Single Sample:

˜30-40 min. With proper labeling and handling to avoid contamination among samples, 3-6 samples can be processed in parallel.
1) Add 300 μL Binding buffer to top of frozen stool and allow to thaw. Once thawed, spin at 400×g for 1 min to pellet solid material.
2) Combine 100 μL stool supernatant, 20 μL proteinase K and 100 μL PBS. Incubate at room temperature for at least 10 min.
3) Add 200 μL Binding Buffer. Invert solution to mix, and filter through the barrel of a tuberculin syringe pre-loaded with a small amount of glass wool. Retain flow through in a new 1.5 ml tube.
4) Add 200 μL isopropanol+50 μL Dynabeads. Incubate for at least 3 min at room temperature. Invert every minute.
5) Pipette up and down in transfer pipette device and then draw total volume into pipette. Apply magnet to steel wool pellet through wall of a 1.5 mL Epp tube and capture beads while expelling liquid. Discard flow through.
6) Remove magnet. Draw up 1.5 mL of Chaotropic Wash Buffer into pipette. Perform 3 passes through pipette. Draw up total volume and apply magnet to steel wool pellet through wall of a 1.5 mL Epp tube. Capture beads and discard flow through.
7) Repeat Step 3, but with Ethanol Wash.
8) Apply magnet to steel wool tip, placing the magnet on the outside wall of the Epp tube and the tip inside of tube containing Rinse Solution. Keeping the magnet adjacent, gently draw up and expel 100 μL rinse solution through steel wool tip in less than 5 seconds. Discard rinse solution.
9) Remove magnet. Draw up 50 μL of Elution Solution and gently mix up and down for at least 1 min. Then draw up total volume and capture the beads in the steel wool using magnet, expelling the eluent into a clean collection tube. Discard transfer pipette and beads. Perform analyses on final eluent.
10) In preliminary efforts, if the final eluent was cloudy, the sample was cleaned through brief centrifugation and removal of supernatant.

Frozen Sputum˜Process and Turnaround Time.

Processing time for a single sample: ˜10-1 5 min. With proper labeling and handling to avoid contamination among samples, 3-6 samples can be processed in parallel.
1) Thaw sample at room temperature.
2) Combine 100 μL sputum+300 μL Binding Buffer in 1.5 mL tube. Invert to Mix. Add 300 μL isopropanol, 3 μL ß-mercaptoethanol, 50 μL Dynabeads. Invert to mix and incubate at room temperature for 3 min. Invert every minute.
3) Pipette up and down in transfer pipette device and then draw total volume into pipette. Apply magnet to steel wool pellet through wall of a 1.5 mL tube and capture beads while expelling liquid. Discard flow through.
4) Remove magnet. Draw up 1.5 mL of Chaotropic Wash Buffer into pipette. Perform 3 passes through pipette. Draw up total volume and apply magnet to steel wool pellet through wall of Epp tube. Capture beads and discard flow through.
5) Repeat Step 3, but with Ethanol Wash.
6) Apply magnet to steel wool tip, placing the magnet on the outside wall of the 1.5 mL tube and the tip inside of tube containing Rinse Solution. Keeping magnet adjacent, gently draw up and expel 100 μL rinse solution through steel wool tip in less than 5 seconds. Discard rinse solution.
7) Remove magnet. Draw up 50 μL of Elution Solution and gently mix up and down for at least 1 minute. Then draw up total volume and capture the beads in the steel wool using magnet, expelling the eluent into a clean collection tube. Discard transfer pipette and beads. Perform analyses on final eluent.

Frozen Whole Blood˜Process and Turnaround Time.

Processing time for a single sample: ˜20-30 min. With proper labeling and handling to avoid contamination among samples, 3-6 samples can be processed in parallel.
1) Thaw sample at room temperature.
2) Combine 100 μL whole blood, 20 μL proteinase K and 100 μL PBS. Incubate for 10 min at 56*C.
3) Add 200 μL Binding Buffer. Invert solution to mix, and filter through the barrel of a tuberculin syringe pre-loaded with a small amount of glass wool. Retain flow through in a new 1.5 ml tube.
4) Add 200 μL isopropanol+50 μL Dynabeads. Incubate for at least 3 min at room temperature. Invert every minute.
5) Pipette up and down in transfer pipette device and then draw total volume into pipette. Apply magnet to steel wool pellet through wall of 1.5 mL tube and capture beads while expelling liquid. Discard flow through.
6) Remove magnet. Draw up 1.5 mL of Chaotropic Wash Buffer into pipette. Perform 3 passes through pipette. Draw up total volume and apply magnet to steel wool pellet through wall of the 1.5 mL tube. Capture beads and discard flow through.
7) Repeat Step 3, but with Ethanol Wash.
8) Apply magnet to steel wool tip, placing the magnet on the outside wall of the Epp tube and the tip inside of tube containing Rinse Solution. Keeping magnet adjacent, gently draw up and expel 100 μL rinse solution through steel wool tip in less than 5 seconds. Discard rinse solution.
9) Remove magnet. Draw up 50 μL of Elution Solution and gently mix up and down for at least 1 minute. Then draw up total volume and capture the beads in the steel wool using magnet, expelling the eluent into a clean collection tube. Discard transfer pipette and beads. Perform analyses on final eluent.

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

1. A kit comprising:
(a) a transfer pipette comprising a reservoir end and an open tip end, wherein the reservoir end of said pipette contains magnetic or paramagnetic beads coated with an analyte binding reagent, and wherein the open end of said pipette comprises steel, copper or aluminum matrix in its lumen; and
(b) a magnet.

2. The kit of claim 1, further comprising one or more of:
(c) a syringe comprising a glass wool-filled tip;
(d) one or more sample collection tubes;
(e) one or more containers comprising processing reagents; and/or
a tuberculin syringe comprising a glass wool filter.

3. The kit of claim 2, wherein the one or more containers contains a dilution buffer, a binding buffer, one or more wash solutions, a rinse solution and/or an elution solution.

4. The kit of claim 1, further comprising a reagent for performing positive and/or negative control reactions.

5. The kit of claim 1, further comprising a device for performing sample collection, such as a syringe, a cup, a tube, a spatula or needle.

6. (canceled)

7. The kit of claim 1, wherein the steel, copper or aluminum matrix is transiently magnetized.

8. The kit of claim 1, further comprising printed instructions for performing sample processing using said kit.

9. The kit of claim 1, wherein said magnetic beads are magnetic or paramagnetic silica beads.

10. The kit of claim 1, wherein said analyte binding reagent is a nucleic acid, antibody, lipid, carbohydrate, or non-antibody protein that binds a target analyte.

11. A transfer pipette comprising a reservoir end and an open tip end, wherein the reservoir end of said pipette contains magnetic or paramagnetic beads coated with an analyte binding reagent, and wherein the open end of said pipette comprises steel, copper or aluminum matrix in its lumen.

12. The transfer pipette of claim 11, wherein the matrix is magnetized.

13. The transfer pipette of claim 11, wherein said magnetic or paramagnetic beads are magnetic or paramagnetic silica beads.

14. The transfer pipette of claim 11, wherein the analyte binding reagent is a nucleic acid, antibody, lipid, carbohydrate, or non-antibody protein that binds a target analyte.

15. The transfer pipette of claim 11, wherein the analyte is a viral, fungal, bacterial or parasitic antigen or an environmental toxin.

16. The transfer pipette of claim 15, wherein the bacterial antigen/nucleic acid is derived from Mycobacterium tuberculosis.

17. The transfer pipette of claim 15, wherein the viral antigen/nucleic acid is derived from Ebola virus, dengue virus, Rift River Valley virus, West Nile virus, Marburg virus, rabies virus, HIV-1, HIV-2, smallpox, hantavirus, influenza virus, Zika virus or rotavirus.

18. The transfer pipette of claim 15, wherein the fungal antigen/nucleic acid is derived from Candida, Histoplasma, Blastomyces, Cryptococcus, Coccidioides, or Paracoccidioides.

19. The transfer pipette of claim 15, wherein the parasitic antigen/nucleic acid is derived from Plasmodium spp., tapeworm, ringworm, heartworm, roundworm, ascaris, whipworm, toxocara, and Acanthamoeba.

20. The transfer pipette of claim 11, further comprising lyophilized reagents for performing a binding or detection assay.

21. A method of purifying an analyte from a solution comprising:
(a) providing a biological or environmental sample;
(b) introducing said sample into a transfer pipette, wherein said transfer pipette comprises a reservoir end and an open tip end, wherein the reservoir end of said pipette contains magnetic or paramagnetic beads coated with an analyte binding reagent, and wherein the open end of said pipette comprises steel, copper or aluminum matrix in its lumen;
(c) expelling the sample from said pipette into a first receptable while placing a magnetic adjacent to said open tip of said pipette;
(d) introducing a first wash solution into said transfer pipette after removing the magnet;
(e) expelling the first wash solution into a second receptacle while:
(i) the tip of said pipette is inserted into said second receptacle; and
(ii) the magnet is placed adjacent to said second receptable,
(f) introducing elution solution into said transfer pipette after removing the magnet; and
(g) expelling the elution solution into a third receptacle.

22-35. (canceled)
